(19) United States
(12) Patent Application Publication
(10) Pub. No.: US 2018/0311451 A1
(43) Pub. Date: Nov. 1, 2018

(54) PRESSURE SUPPORT SYSTEM AND METHOD OF CONTROLLING AN AUDIBLE OUTPUT OF SAME

(71) Applicant: KONINKLIJKE PHILIPS N.V., EINDHOVEN (NL)

(72) Inventors: ELIZABETH POWELL MARGARIA, PITTSBURGH, PA (US); EUGENE NELSON SCARBERRY, TRAFFORD, PA (US); ROBERT WILLIAM BAIKO, PITTSBURGH, PA (US)

(21) Appl. No.: 15/768,366

(22) PCT Filed: Oct. 8, 2016
(86) PCT No.: PCT/IB2016/056038

§ 371 Date: Apr. 13, 2018

Related U.S. Application Data
(60) Provisional application No. 62/243,732, filed on Oct. 20, 2015.

(51) Int. Cl. A61M 16/00; A61M 16/08; A61M 16/06; A61M 16/16

(52) U.S. Cl. CPC A61M 16/0003; A61M 16/0875; A61M 16/0057; A61M 2205/43; A61M 16/16; A61M 2205/42; A61M 16/06

(57) ABSTRACT

A pressure support system includes a gas flow generator; a patient interface device structured to be secured to a patient; a gas delivery conduit structured to couple the gas flow generator to the patient interface device; and a passive noise system provided on at least one of the gas flow generator, the patient interface device, and the gas delivery conduit, the passive noise system being structured to utilize a flow of gas to alter a base audible output of the pressure support system.

### CROSS-REFERENCE TO RELATED APPLICATIONS

This patent application claims the priority benefit under 35 U.S.C. § 119(e) of U.S. Provisional Application No. 62/243,732 filed on Oct. 20, 2015, the contents of which are herein incorporated by reference.

### BACKGROUND OF THE INVENTION

### 1. Field of the Invention

The present invention relates to non-invasive ventilation and pressure support systems wherein a patient interface device is used to deliver a flow of breathing gas to a patient. The present invention also relates to methods of controlling an audible output of a pressure support system.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping. Non-invasive ventilation and pressure support therapies as just described involve a gas flow generator to produce a flow of breathing gas, and the placement of a patient interface device including a mask component on the face of a patient. The gas flow generator produces positive air pressure by taking air in from the surroundings and spinning a fan to push the air out of the machine, through a delivery conduit, and into the patient interface device to be delivered to the patient.

PAP therapy machines by their very nature generate audible outputs (e.g., noises, sounds, or tones that have loudness and frequency). For example, gas flow through conduits generates noises, and the fan of the gas flow generator typically generates noises. Furthermore, as the patient inhales and exhales, the speed of the fan increases or decreases to maintain therapy pressure. These changes in speed create variances in the audible output, which can likewise be distracting to the patient. Moreover, the interface device itself includes openings for carbon dioxide to be expelled. Gas flow through these openings creates additional noises, which can also be distracting to the patient who is trying to sleep. In order for therapy to be delivered effectively, it is desirable for audible outputs to be conducive to sleeping, so that the patient is not disturbed.

### SUMMARY OF THE INVENTION

Accordingly, it is an object of the present invention to provide a pressure support system that includes a gas flow generator, a patient interface device structured to be secured to a patient, a gas delivery conduit structured to couple the gas flow generator to the patient interface device, and a passive noise system provided on at least one of the gas flow generator, the patient interface device, and the gas delivery conduit, the passive noise system being structured to utilize a flow of gas to alter a base audible output of the pressure support system.

It is yet another object of the present invention to provide a method of controlling an audible output of a pressure support system. The pressure support system has a base audible output associated therewith and includes a gas flow generator, a patient interface device structured to be secured to a patient, a gas delivery conduit structured to couple the gas flow generator to the patient interface device, and a passive noise system provided on at least one of the gas flow generator, the patient interface device, and the gas delivery conduit. The method includes the steps of: producing the base audible output with the pressure support system; and altering the base audible output with the passive noise system by utilizing a flow of gas, thereby producing an altered audible output.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a simplified view of a pressure support system, in accordance with a non-limiting embodiment of the disclosed concept;

FIG. 2 is a simplified exploded view of a portion of the pressure support system of FIG. 1;

FIG. 3 is a simplified top plan view of a cuff member and a grate member for the pressure support system of FIG. 1;

FIG. 4 is a simplified view of a pressure support system, in accordance with another non-limiting embodiment of the disclosed concept;

FIG. 5 is a simplified enlarged view of a portion of the pressure support system of FIG. 4;

FIG. 6 is a simplified section view of the portion of the pressure support system of FIG. 5, taken along line AA of FIG. 5;

FIG. 7 is a simplified view of a pressure support system, shown with portions removed in order to see hidden structures, in accordance with another non-limiting embodiment of the disclosed concept;

FIGS. 8 and 9 are simplified enlarged views of the gas flow generator for the pressure support system of FIG. 7, shown with the movable member and the fixed member, and with the movable member in different positions with respect to the fixed member;

FIG. 10 is a simplified view of a pressure support system, in accordance with another non-limiting embodiment of the disclosed concept;

FIG. 11 is a simplified view of a portion of the gas flow generator for the pressure support system of FIG. 10, modified to show the outlet of the humidifier;

FIG. 12 is an enlarged view of the portion of the gas flow generator of FIG. 11, and shown with the movable member and the fixed member; and

FIG. 13 is a simplified view of a pressure support system, in accordance with another non-limiting embodiment of the disclosed concept.

### DETAILED DESCRIPTION OF EXEMPLARY EMBODIMENTS

As used herein, the singular form of “a,” “an,” and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As employed herein, the term “number” shall mean one or an integer greater than one (i.e., a plurality).

As employed herein, the term “base audible output” shall mean an audible output (i.e., sound and/or noise) of a pressure support system that does not employ a passive noise system as described hereinbelow. As employed herein, the term “altered audible output” shall mean an audible output (i.e., sound and/or noise) of a pressure support system that has been altered in at least one respect. For example and without limitation, a base audible output that has been altered by an exhalation conduit and/or a masking apparatus is an altered audible output.

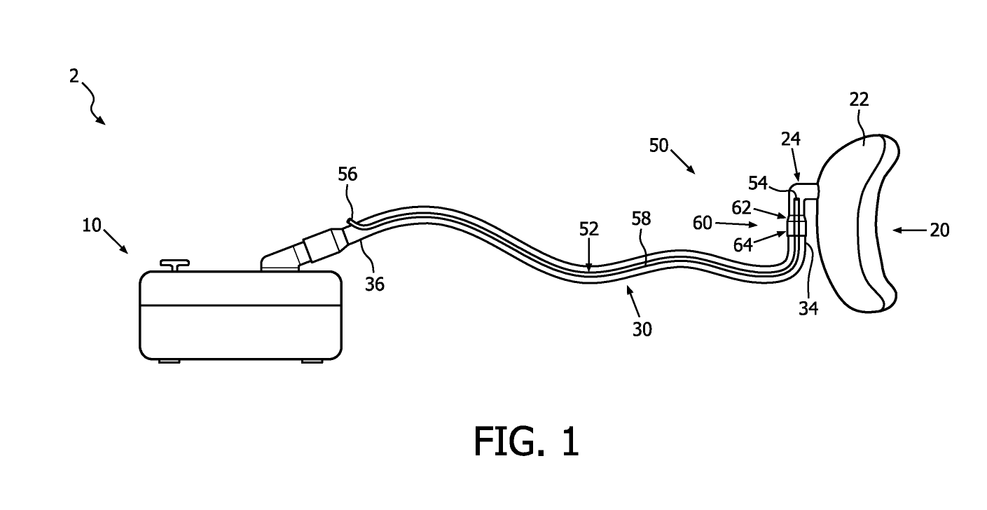

FIG. 1 shows a simplified view of a pressure support system 2 in accordance with a non-limiting embodiment of the disclosed concept. Pressure support system 2 includes a gas flow generator 10, a patient interface device 20 structured to be secured to a patient, and a gas delivery conduit (e.g., hose 30) coupling gas flow generator 10 to patient interface device 20. During pressure support therapy with a patient, pressure support system 2 produces a base audible output (i.e., sound or noise). The base audible output has a loudness and a frequency that each vary over time. As discussed above, certain audible outputs are often very disturbing to patients who are trying to remain asleep. Accordingly, as will be discussed in greater detail hereinbelow, pressure support system 2 advantageously includes a passive noise system 50 that is structured to utilize a flow of gas to passively alter the base audible output (i.e., alter the loudness and/or the frequency) of pressure support system 2, thereby resulting in an environment that is more conducive to sleeping.

Continuing to refer to FIG. 1, passive noise system 50 includes an exhalation conduit 52 and a masking apparatus 60 which, as described herein, alone or together alter the base audible output. Exhalation conduit 52 has a first distal end portion 54 coupled to patient interface device 20, a second distal end portion 56 located opposite and distal first distal end portion 54, and a body portion 58 located therebetween. Masking apparatus 60 includes a movable member (e.g., annular grate member 62) and a fixed member (e.g., cuff member 64) coupled to grate member 62. Grate member 62 and cuff member 64 are coupled to and in fluid communication with hose 30 and patient interface device 20. Patient interface device 20 has a mask component 22 and an elbow member 24 coupled thereto.

Hose 30 includes a first distal end portion 34 and a second distal end portion 36 located opposite and distal first distal end portion 34. Second distal end portion 36 is coupled to gas flow generator 10. First distal end portion 34 and grate member 62 are coupled to cuff member 64 and elbow member 24. As shown, exhalation conduit 52 extends in a direction away from patient interface device 20. More specifically, first distal end portion 54 is located internal (i.e., is enclosed or surrounded by) elbow member 24, body portion 58 is located internal (i.e., is enclosed or surrounded by) hose 30, and second distal end portion 56 is located external (i.e., is outside of) hose 30. Additionally, second distal end portion 56 is located proximate second distal end portion 36 and external with respect thereto.

In this manner, expelled gases from the patient are structured to be diverted from first distal end portion 54 through exhalation conduit 52 (e.g., and also through hose 30) before exiting second distal end portion 56. Additionally, exhalation conduit 52 is made of a relatively soft and flexible elastomeric material. In this manner, exhalation conduit 52 is advantageously structured to bend responsive to movement of hose 30. The inventors have discovered that employing exhalation conduit 52 advantageously results in improved results. More specifically, the inventors have discovered that exhalation conduit 52 causes the volume, or loudness, of the base audible output of pressure support system 2 to advantageously be reduced as well as be dampened during pressure support therapy. That is, in addition to having the base audible output be made quieter during therapy, variations in loudness are significantly minimized. Stated differently, exhalation conduit 52 advantageously allows the base audible output of pressure support system 2 to be altered, resulting in an altered audible output that is of relatively steady and quiet volume.

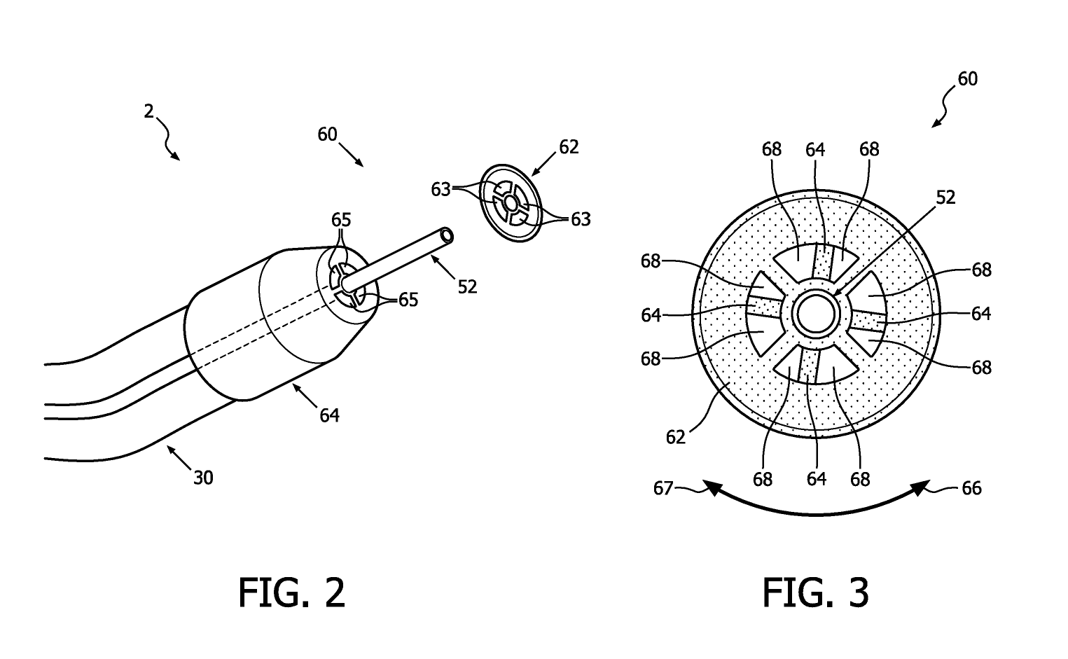

FIG. 2 shows an exploded view of grate member 62 and cuff member 64. As shown, grate member 62 has a number of openings 63 and cuff member 64 has a number of openings 65. During pressure support therapy, breathing gas is structured to flow through openings 63,65 before being delivered to the patient. Responsive to the breathing gas (i.e., gas from gas flow generator 10 that flows through hose 30) flowing through openings 63,65, grate member 62 and cuff member 64 create a masking noise (i.e., a broadband white noise having a relatively constant average frequency) with the flow of breathing gas that is structured to mask a frequency of the altered audible output (i.e., the audible output resulting from the base audible output being altered by exhalation conduit 52), thereby producing a combined audible output that is more conducive to sleeping.

The masking noise created by grate member 62 and cuff member 64 responsive to the flow of gas being passed therethrough has a frequency that is dependent upon the relative positions of grate member 62 and cuff member 64. More specifically, and with reference to FIG. 3, grate member 62 is structured to move independently with respect to cuff member 64, thereby allowing different masking frequencies to be created. That is, grate member 62 is structured to rotate about exhalation conduit 52 in either a first direction 66 or a second direction 67 opposite first direction 66. As shown in the depicted position of FIG. 3, grate member 62 substantially overlays cuff member 64 such that a number of combined openings 68 are formed by openings 63,65. When grate member 62 rotates in either of directions 66,67, the size of openings 68 changes, which changes the frequency of the masking noise created by grate member 62 and cuff member 64.

Accordingly, masking apparatus 60 utilizes a flow of gas to create a masking noise to passively mask a frequency of the altered audible output (i.e., the audible output resulting from the base audible output being altered by exhalation conduit 52) of pressure support system 2, thereby producing a combined audible output that is more pleasant and conducive to sleeping. As stated hereinabove, a significant drawback of known pressure support systems is that the audible outputs often have varying frequencies and at times relatively high pitched frequencies, both of which can significantly inhibit the ability of the patient to effectively sleep at night.

During pressure support therapy, the altered audible output (i.e., the audible output resulting from the base audible output being altered by exhalation conduit 52) has a frequency. When the flow of gas flows through masking apparatus 60, the masking noise that is created has an average frequency that is proximate a frequency of the altered audible output, thus masking the variations in frequency of the altered audible output and producing the combined audible output that is a relatively steady broadband sound. Furthermore, masking apparatus 60 advantageously allows different frequencies of masking noise to be produced by the flow of gas. For example and without limitation, if the patient desires to create a masking noise with a different frequency to mask the frequency of the altered audible output, rotation of grate member 62 in directions 66,67 will advantageously cause the size of openings 68 to change, thereby changing the frequency of the masking noise.

Although pressure support system 2 has been described in association with exhalation conduit 52 and masking apparatus 60, it will be appreciated that a similar suitable alternative pressure support system could employ only one of exhalation conduit 52 and masking apparatus 60, without departing from the scope of the disclosed concept. Thus, in an alternative implementation, masking apparatus 60 could create a masking noise with the flow of gas to mask a frequency of the base audible output (i.e., in a pressure support system without exhalation conduit 52), thereby producing a combined audible output of the base audible output and the masking noise.

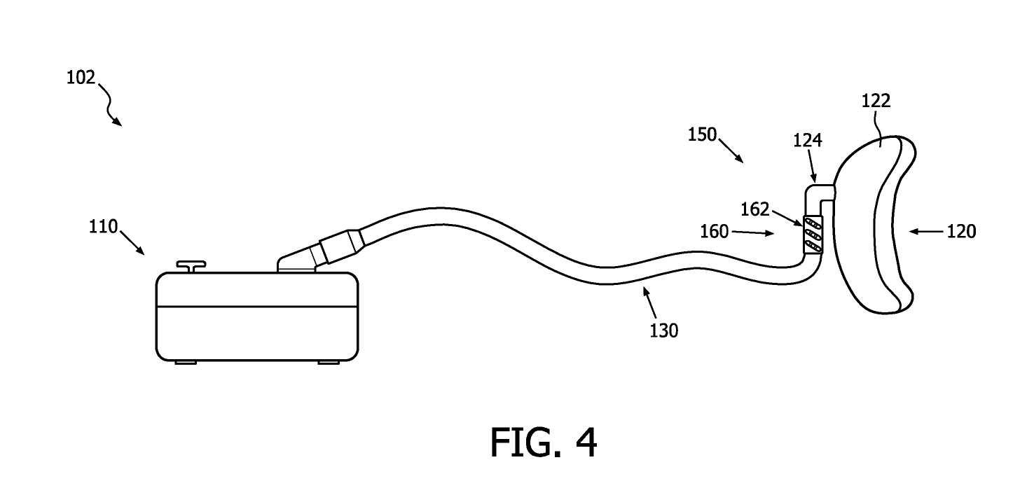

FIG. 4 shows a simplified view of an alternative pressure support system 102 according to an alternative exemplary embodiment. As seen in FIG. 4, alternative pressure support system 102 includes many components that are similar to pressure support system 2 (FIG. 1). For example, pressure support system 102 includes a gas flow generator 110, a patient interface device 120 structured to be secured to a patient, and a gas delivery conduit (e.g., hose 130). Pressure support system 102 also includes a passive noise system 150 that includes a masking apparatus 160. Masking apparatus 160 creates a masking noise with a flow of gas to mask a frequency of the altered audible output of pressure support system 102, thereby producing a combined audible output that is more conducive to sleeping.

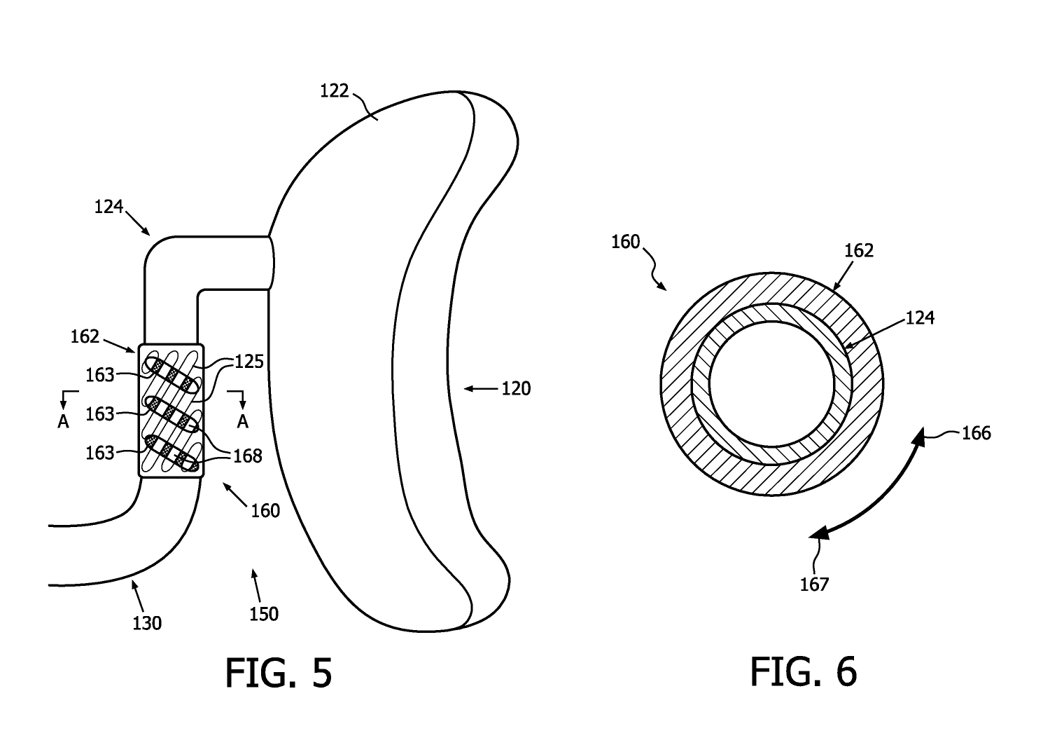

Patient interface device 120 includes a mask component 122 and an elbow assembly coupled thereto. Masking apparatus 160 includes the elbow assembly of patient interface device 120. The elbow assembly includes elbow member 124 and a cylindrical-shaped grate member 162 coupled thereto. As shown in the enlarged view of FIG. 5, elbow member 124 includes a number of openings 125 and grate member 162 includes a number of openings 163. Additionally, elbow member 124 substantially extends through grate member 162 such that a number of combined openings 168 are formed by openings 125,163. In other words, grate member 162 is generally a hollow sleeve located on elbow member 124. When breathing gas exits through openings 168, a masking noise (i.e., a broadband white noise having a relatively constant average frequency) is created to mask a frequency of the altered audible output of pressure support system 102, thereby producing the combined audible output. Referring to the section view of FIG. 6, grate member 162 is a movable member that is structured to move independently with respect to elbow member 124, which is a fixed member. More specifically, grate member 162 is structured to rotate about elbow member 124 in either a first direction 166 or a second direction 167 opposite first direction 166. As a result of movement by grate member 162 in either of directions 166,167, the size of openings 168 changes, thereby changing the frequency of the masking noise that is created with the flow of gas.

Accordingly, advantages associated with masking apparatus 60 of pressure support system 2 likewise apply to masking apparatus 160 of pressure support system 102. Additionally, as shown and described, the structure of masking apparatus 160 is different from the structure of masking apparatus 60 (FIGS. 1-3). It will be appreciated that this provides the additional advantages of easily allowing for an interchangeable cuff member (i.e., grate member) and easily allowing for expelled gases to be directed toward a given direction (i.e., away from a bed partner).

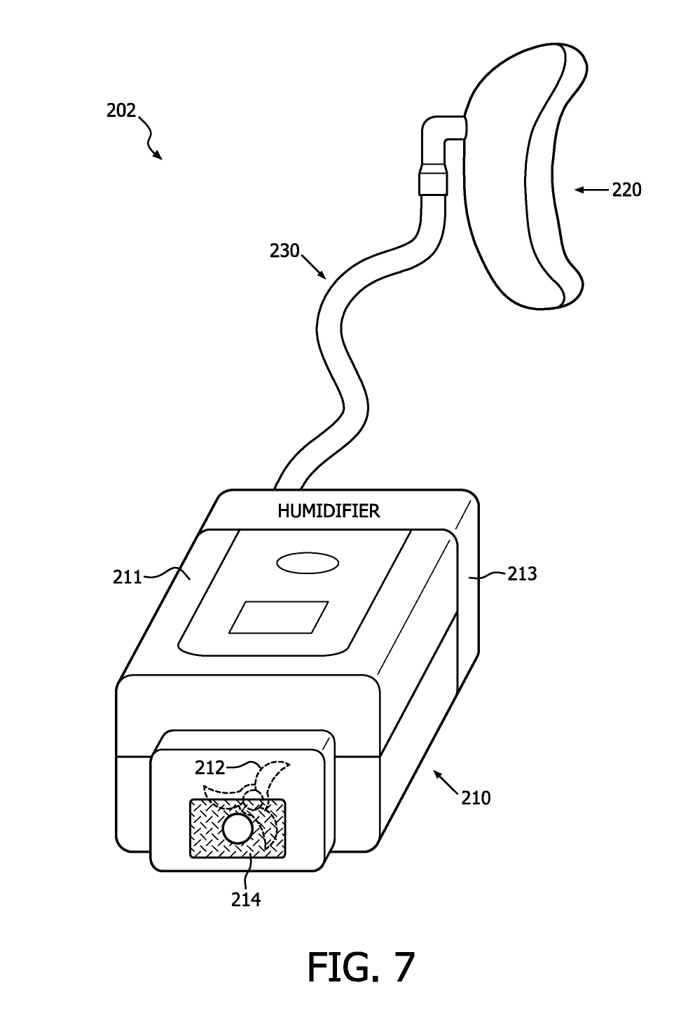

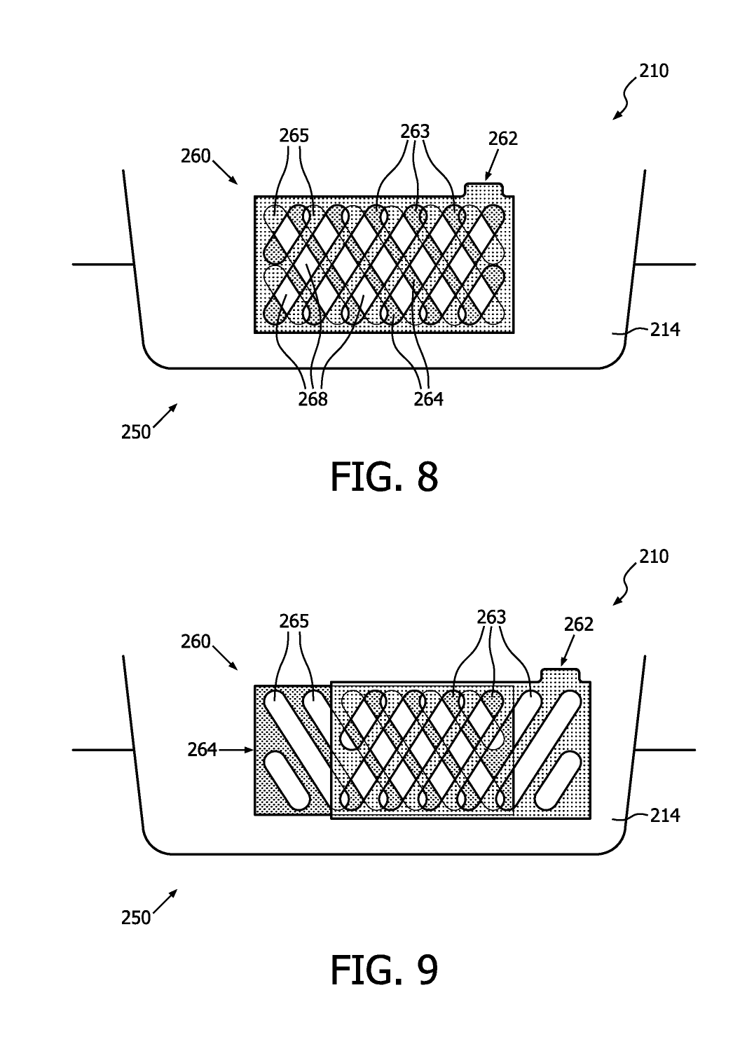

FIG. 7 shows a simplified view of an alternative pressure support system 202 according to an alternative exemplary embodiment. As seen FIG. 7, alternative pressure support system 202 includes many components that are similar to pressure support system 2 (FIG. 1). For example, pressure support system 202 includes a gas flow generator 210, a patient interface device 220 structured to be secured to a patient, and a gas delivery conduit (e.g., hose 230). As shown in FIG. 7, gas flow generator 210 includes a housing 211, and a fan member 212 and a humidifier 213 that are coupled to housing 211. Housing 211 has an inlet portion 214 located proximate fan member 212. Fan member 212 is structured to pull a flow of gas from outside gas flow generator 210 through inlet portion 214 in order to be delivered to humidifier 213. Pressure support system 202 also includes a passive noise system 250 (not shown in FIG. 7 in order to see inlet portion 214, but shown in FIGS. 8 and 9) located on gas flow generator 210.

FIGS. 8 and 9 show simplified views of an enlarged portion of gas flow generator 210. As shown, passive noise system 250 includes a masking apparatus 260 that is coupled to inlet portion 214. Masking apparatus 260 includes a movable member (e.g., grate member 262) and a fixed member 264 each coupled to one another and to inlet portion 214. Grate member 262 has a number of openings 263 and fixed member 264 has a number of openings 265. Referring to FIG. 8, grate member 262 substantially overlays (i.e., is generally parallel with and located directly on top of when viewed from a top plan view) fixed member 264 such that a number of combined openings 268 are formed by openings 263,265. Because grate member 262 and fixed member 264 are coupled to inlet portion 214, fan member 212 is structured to pull a flow of gas through openings 268 before delivering the gas to humidifier 213.

When the flow of gas passes through openings 268, grate member 262 and fixed member 264 create a masking noise (i.e., a broadband white noise having a relatively constant average frequency) to mask a frequency of the base audible output of pressure support system 202. Additionally, grate member 262 is structured to move independently with respect to fixed member 264. For example and without limitation, grate member 262 is in a different position with respect to fixed member 264 in FIG. 9 than in FIG. 8. As such, in the depicted position of FIG. 9, the flow of gas will have a number of relatively large openings 265 to flow through, as well as a number of combined openings. Thus, the frequency of masking noise created by a flow of gas through masking apparatus 260 would be different in the respective positions of FIG. 8 and FIG. 9. Accordingly, it will be appreciated that advantages associated with masking apparatuses 60, 160 (FIGS. 1-6) likewise apply to masking apparatus 260 of passive noise system 250.

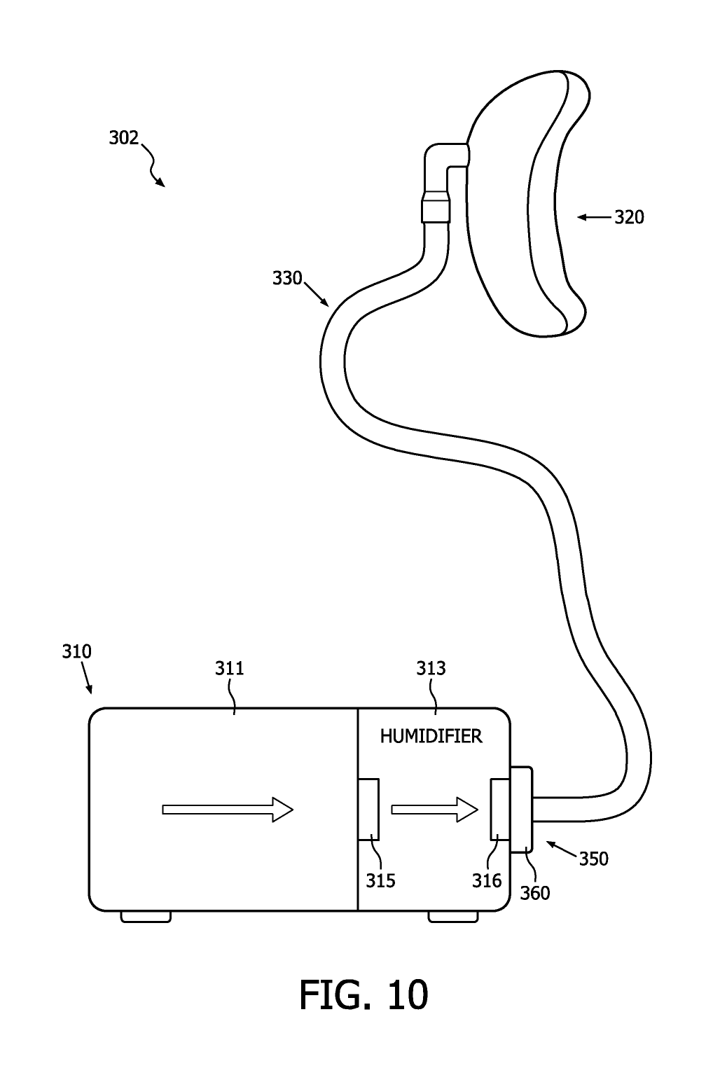

FIG. 10 shows a simplified view of an alternative pressure support system 302 according to an alternative exemplary embodiment. As seen in FIG. 10, alternative pressure support system 302 includes many components that are similar to pressure support system 2 (FIG. 1). For example, pressure support system 302 includes a gas flow generator 310, a patient interface device 320 structured to be secured to a patient, and a gas delivery conduit (e.g., hose 330). Gas flow generator 310 includes a housing 311 and a humidifier 313 coupled thereto. Humidifier 313 has an inlet portion 315 and an outlet portion 316. Additionally, pressure support system 302 has a passive noise system 350 that includes a masking apparatus 360 coupled to outlet portion 316.

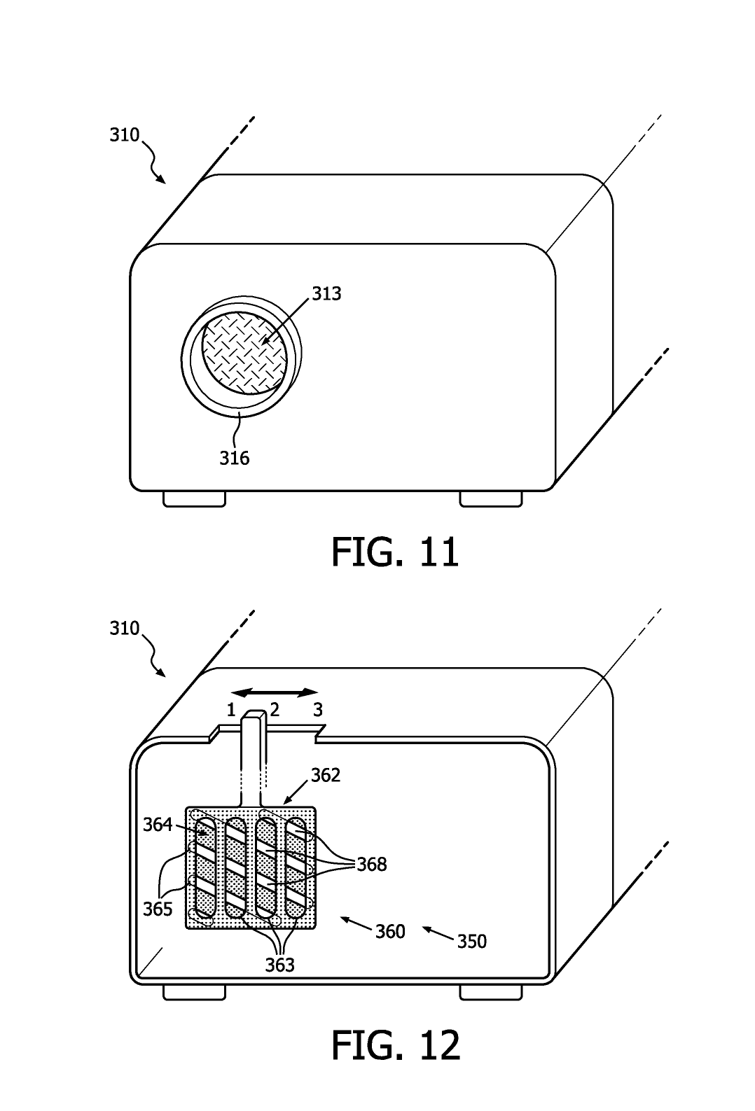

FIG. 11 shows a simplified view of gas flow generator 310 with portions removed in order to see outlet portion 316. FIG. 12 shows a simplified view of a portion of gas flow generator 310 with masking apparatus 360. Masking apparatus 360 functions substantially the same as and affords similar benefits as masking apparatuses 60, 160, 260 (FIGS. 1-6 and 8-9). More specifically, masking apparatus 360 includes a movable member (e.g., grate member 362) and a fixed member 364 each coupled to one another and to outlet portion 316. Grate member 362 and fixed member 364 each have respective openings 363, 365 and substantially overlay (i.e., are parallel to one another and located on top of one another when viewed from a top plan view) one another such that combined openings 368 are formed by openings 363, 365. When a flow of gas passes through outlet portion 316 of humidifier 313, the flow of gas passes through openings 368, thereby creating a masking noise (i.e., a broadband white noise having a relatively constant average frequency) to mask a frequency of the base audible output of pressure support system 302. Additionally, grate member 362, like grate members 62, 162, 262, is structured to move independently with respect to fixed member 364, thereby changing the size of the openings through which the flow of gas passes. As a result, the frequency of the masking noise that is created, like the frequency of the masking noises created by the masking apparatuses 60, 160, 260, is advantageously able to be different depending on the desired audible output of the patient. Accordingly, advantages associated with masking apparatuses 60, 160, 260 likewise apply to masking apparatus 360 in pressure support system 302.

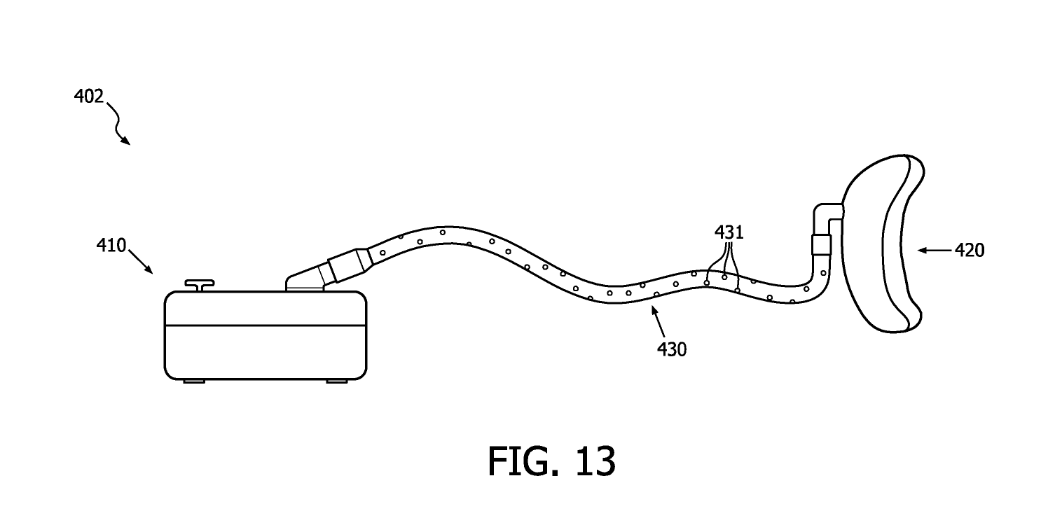

FIG. 13 shows a simplified view of an alternative pressure support system 402 according to an alternative exemplary embodiment. As shown in FIG. 13, alternative pressure support system 402 includes many components that are similar to pressure support system 2 (FIG. 1). For example, pressure support system 402 includes a gas flow generator 410, a patient interface device 420 structured to be secured to a patient, and a gas delivery conduit (e.g., hose 430). Hose 430 has a plurality of thru holes 431. As a result, hose 430 is structured to create a masking noise (i.e., a broadband white noise having a relatively constant average frequency) responsive to a flow of gas (i.e., a flow of gas from gas flow generator 410 being delivered to patient interface device 420 through hose 430) being passed through thru holes 431. In other words, hose 430 is a passive noise system that is structured to create a masking noise that has a frequency to mask a second frequency of the base audible output of pressure support system 402, thereby producing a combined audible output that is more pleasant and conducive to sleeping.

It will be appreciated that a method of controlling an audible output of pressure support system 2, 102, 202, 302, 402 includes the steps of producing the base audible output with respective pressure support system 2, 102, 202, 302, 402; and altering the base audible output with respective passive noise system 50, 150, 250, 350 by utilizing a flow of gas, thereby producing an altered audible output. The altering step may, without limitation, include diverting the flow of gas from a respective patient interface device 20 through an exhalation conduit 52. The altering step may also, without limitation, include passing the flow of gas through respective openings 63, 65, 125, 163, 263, 265, 363, 365 of respective fixed member 64, 124, 264, 364 and respective movable member 62, 162, 262, 362 in order to create a masking noise, the masking noise and the base audible output combining to produce the altered audible output.

Passive noise systems 50, 150, 250, 350 and hose 430 advantageously utilize flows of gas to alter the base audible outputs of respective pressure support systems 2, 102, 202, 302, 402. This is distinct from prior art active noise systems (not shown) in which speakers or other active noise producing mechanisms create cancelation frequencies based on detected frequencies. Such active noise cancelation systems would add undesirable costs to a pressure support system and present potential problems in the event that sensors malfunction. Accordingly, the base audible outputs of pressure support systems 2, 102, 202, 302, 402 are advantageously able to be reliably altered (i.e., changed into an altered audible output that is more conducive to sleeping) by passively relying on flows of gas.

Although the disclosed base audible output altering concept has been described in association with pressure support systems 2, 102, 202, 302, 402, it will be appreciated that suitable alternative pressure support systems are within the scope of the disclosed concept. More specifically, any of the elements (i.e., exhalation conduit 52, masking apparatuses 60, 160, 260, 360, and hose 430) of pressure support systems 2, 102, 202, 302, 402 that alter the respective base audible outputs may be employed in isolation or in any possible combination. That is, respective passive noise systems 50, 150, 250, 350 may be provided on at least one of gas flow generators 10, 110, 210, 310, 410, patient interface devices 20, 120, 220, 320, 420, and hoses 30, 130, 230, 330. Also, hose 430 may be substituted for hoses 30, 130, 230, 330 in pressure support systems 2, 102, 202, 302. Additionally, although the masking apparatuses 60, 160, 260,360 and hose 430 have been illustrated with the depicted shapes of openings 63, 65, 125, 163, 263, 265, 363, 365 and thru holes 431, it will be appreciated that a similar suitable masking apparatus and/or hose may have respective openings and respective thru holes having any desired shape in order to create a masking noise with any desired masking frequency, without departing from the scope of the disclosed concept.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A pressure support system comprising:
a gas flow generator;
a patient interface device structured to be secured to a patient,
a gas delivery conduit structured to couple the gas flow generator to the patient interface device; and
a passive noise system provided on the gas flow generator, the passive noise system being structured to utilize a flow of gas to alter a base audible output of the pressure support system, wherein the passive noise system comprises a masking apparatus provided on the gas flow generator, and wherein the masking apparatus is structured to create a masking noise with the flow of gas to mask a frequency of the base audible output, thereby producing a combined audible output.

2-7. (canceled)

8. The pressure support system according to claim 1, wherein the masking apparatus comprises a fixed member and a movable member coupled thereto; wherein the fixed member and the movable member each have a number of openings; wherein the fixed member and the movable member are structured to create the masking noise responsive to the flow of gas being passed through the number of openings of the fixed member and the movable member; and wherein the movable member is structured to move independently with respect to the fixed member.

9. (canceled)

10. The pressure support system according to claim 8, wherein the gas flow generator comprises a housing, a fan member, and a humidifier; wherein the fan member and the humidifier are coupled to the housing; wherein the housing has an inlet portion disposed proximate the fan member; wherein the fixed member and the movable member are each coupled to the inlet portion; and wherein the fan member is structured to pull the flow of gas through the openings of the fixed member and the movable member before delivering the flow of gas to the humidifier.

11. The pressure support system according to claim 8, wherein the gas flow generator comprises a housing and a humidifier coupled thereto; wherein the humidifier has an inlet portion and an outlet portion; and wherein the fixed member and the movable member are coupled to the outlet portion.

12. (canceled)

13. A method of controlling an audible output of a pressure support system, the pressure support system having a base audible output associated therewith and comprising a gas flow generator, a patient interface device structured to be secured to a patient, a gas delivery conduit structured to couple the gas flow generator to the patient interface device, and a passive noise system provided on the gas delivery conduit, the passive noise system comprising a masking apparatus provided on the gas flow generator, the method comprising the steps of:
producing the base audible output with the pressure support system; and
altering the base audible output with the masking apparatus by utilizing a flow of gas, thereby producing an altered audible output.

14. (canceled)

15. The method according to claim 13, wherein the masking apparatus comprises a fixed member and a movable member coupled thereto; wherein the fixed member and the movable member each have a number of openings; and wherein the altering step comprises:
passing the flow of gas through the number of openings of the fixed member and the movable member in order to create a masking noise, the masking noise and the base audible output combining to produce the altered audible output.
